(19) United States
(12) Patent Application Publication
(10) Pub. No.: US 2021/0275464 A1
(43) Pub. Date: Sep. 9, 2021

(54) BIOMEMBRANE-COVERED NANOPARTICLES (BIONPS) FOR DELIVERING ACTIVE AGENTS TO STEM CELLS

(71) Applicants: Eleftherios T. Papoutsakis, Newark, DE (US); Emily Day, Landenburg, PA (US); Erica Winter, West Chester, PA (US); Jenna Harris, Cummington, MA (US); Chen-Yuan Kao, Newark, DE (US); Samik Das, Wilmington, DE (US)

(72) Inventors: Eleftherios T. Papoutsakis, Newark, DE (US); Emily Day, Landenburg, PA (US); Erica Winter, West Chester, PA (US); Jenna Harris, Cummington, MA (US); Chen-Yuan Kao, Newark, DE (US); Samik Das, Wilmington, DE (US)

(73) Assignee: University of Delaware, Newark, DE (US)

(21) Appl. No.: 17/327,932

(22) Filed: May 24, 2021

Related U.S. Application Data
(63) Continuation of application No. PCT/US2019/063685, filed on Nov. 27, 2019.
(60) Provisional application No. 62/772,311, filed on Nov. 28, 2018.

(51) Int. Cl. A61K 9/51; A61K 45/06

(52) U.S. Cl. CPC A61K 9/5184; B82Y 5/00; A61K 45/06; A61K 9/5153

(57) ABSTRACT

The present invention provides bio-nanoparticles (BioNPs) for delivering an active agent into hematopoietic stem & progenitor cells (HSPCs). Each BioNP comprises a core and a biological membrane covering the core, which comprises the active agent and a polymer. The biological membrane comprises a phospholipid bilayer and one or more surface proteins of a megakaryocyte (Mk). The active agent remains active after being delivered into the HSPC. Also provided are methods for preparing the BioNPs and uses of the BioNPs for targeted delivery of an active agent into HSPCs and/or treating or preventing a disease or condition in a subject in need thereof.

### CROSS-REFERENCE TO RELATED APPLICATION

This application is a Continuation Application of International Application No. PCT/US2019/063685, filed Nov. 27, 2019, claiming priority to United States Provisional Application No. 62/772,311, filed Nov. 28, 2018, the contents of which are incorporated herein by reference in their entireties for all purposes.

### REFERENCE TO U.S. GOVERNMENT SUPPORT

This invention was made with government support under Grant No. 1752009 from the National Science Foundation. The United States has certain rights in the invention.

### FIELD OF THE INVENTION

The invention relates generally to biomembrane-covered nanoparticles (BioNPs) comprising active agents and uses thereof for targeted delivery of the active agents into hematopoietic stem & progenitor cells (HSPCs) with high specificity and controlled release of the active agents from the BioNPs in the HSPCs.

### BACKGROUND OF THE INVENTION

Hematopoietic stem & progenitor cells (HSPCs) are located in the bone marrow and possess the ability to self-renew or differentiate into any blood lineage cell. Their ability to differentiate into blood-related cells makes HSPCs ideal candidates for therapeutic manipulation through gene regulation or other means. Indeed, controlling HSPC function holds “formidable promise . . . that may transform medical practice.” However, cargo delivery to HSPCs is a long-standing problem. Current delivery methods in the form of viral vectors (lentivirus, adeno-associated virus) have limited loading capacity, poor DNA insertion, and produce too much cytotoxicity. Non-viral vectors (naked plasmids, siRNAs, etc.) have short half-lives when introduced into the bloodstream, as they are recognized by the innate immune system, and they are also easily degraded or have limited cell membrane penetration. To successfully deliver therapeutic or imaging cargo to HSPCs a delivery system must target HSPCs specifically while protecting the cargo. Megakaryocyte-derived microparticles (MkMPs), which are one type of extracellular vesicles (EVs), have previously been shown to specifically target and enter HSPCs through receptor-meditated endocytosis. Although nanoparticles wrapped in membranes derived from red blood cells, platelets, or cancer cells have been previously described and utilized for hydrophobic drug delivery, no studies have reported the use of nanoparticles wrapped in Mk-derived membranes for targeted delivery of active agents to HSPCs. Nor have any studies reported the delivery of hydrophilic cargo such as nucleic acids to HSPCs with using membrane-covered nanoparticles.

There remains a need for highly reproducible nanoparticles for targeted delivery and controlled release of both hydrophobic and hydrophilic active agents into hematopoietic stem & progenitor cells (HSPCs) with high specificity.

### SUMMARY OF THE INVENTION

The present invention relates to bio-nanoparticles (BioNPs) for delivering an active agent into hematopoietic stem & progenitor cells (HSPCs) and uses thereof.

A bio-nanoparticle for delivering an active agent into a hematopoietic stem & progenitor cell (HSPC) is provided. The bio-nanoparticle comprises a core and a biological membrane covering the core. The core comprises the active agent and a polymer. The biological membrane comprises a phospholipid bilayer and one or more surface proteins of megakaryocyte cells (Mks or Mk cells). The active agent remains active after being delivered into the HSPC. The biological membrane may be adhered to the core by an electrostatic interaction.

The biological membrane may be prepared from a megakaryocyte (Mk), megakaryocytic microparticle (MkMP) or megakaryocytic extracellular vesicle. The megakaryocyte (Mk), megakaryocytic microparticle or megakaryocytic extracellular vesicle may be prepared from a hematopoietic stem & progenitor cell (HSPC). The megakaryocyte (Mk), megakaryocytic microparticle or megakaryocytic extracellular vesicle may be prepared from a human megakaryocyte cell line. The biological membrane may be prepared from a megakaryocyte (Mk) and the bio-nanoparticle lacks a cytosolic, nuclear or mitochondrial component of the Mk.

The Mk surface proteins may be selected from the group consisting of CD62P, VLA-4 (CD49d), CD41, CD150, CXCR4, thrombopoietin (TPO) receptor, c-kit, CD34, CD105 (endoglin), CD31 (9PECAM-1), JAM-A, Tie-2, KDR (VEGF receptor 2) and a combination thereof. In one embodiment, the one or more surface proteins may comprise CD41. In another embodiment, the one or more surface proteins comprise VLA-4 (CD49d).

The polymer may be poly(lactic-co-glycolic acid) (PLGA).

The active agent may be hydrophobic and the core may be prepared from a single-emulsion.

The active agent many be hydrophilic and the core may be prepared from a double-emulsion.

The active agent may be selected from the group consisting of an imaging agent, a therapeutic agent, and a combination thereof. The imaging agent may be selected from the group consisting of fluorophores, MRI contrast agents, CT contrast agents, ultrasound contrast agents, and combinations thereof. The therapeutic agent may be a nucleic acid molecule selected from the group consisting of siRNA, miRNA, DNA, and a combination thereof. The DNA may be a single-stranded DNA. The therapeutic agent may be selected from the group consisting of chemotherapeutics, HSPC mobilizing agents, and a combination thereof. The therapeutic agent may be a chemotherapeutic. The core may further comprise an excipient.

A method for preparing a bio-nanoparticle for delivering an active agent into a hematopoietic stem & progenitor cell (HSPC) is also provided. The preparation method may comprise coating a core with a biological membrane at an effective weight ratio for forming a bio-nanoparticle such that the core comprises the active agent and a polymer, and the biological membrane comprises two layers of phospholipids and one or more surface proteins of a megakaryocyte (Mk). The active agent remains active after being delivered into the HSPC.

The preparation method may further comprise preparing the biological membrane from a megakaryocyte (Mk), megakaryocytic microparticle or megakaryocytic extracellular vesicle. The preparation method may further comprise preparing the megakaryocyte (Mk), megakaryocytic microparticle or megakaryocytic extracellular vesicle from a hematopoietic stem & progenitor cell (HSPC). The preparation method may further comprise preparing the megakaryocyte (Mk), megakaryocytic microparticle or megakaryocytic extracellular vesicle from a human megakaryocyte cell line. The preparation method may further comprise preparing the biological membrane from a megakaryocyte (Mk) after one or more components of the Mk are removed from the Mk, and the one or more components are selected from the group consisting of cytosolic, nuclear and mitochondrial components.

The preparation method may further comprise adhering the biological membrane to the core by an electrostatic interaction.

According to the preparation method, the one or more surface proteins may be selected from the group consisting of CD62P, VLA-4 (CD49d), CD41, CD150, CXCR4, thrombopoietin (TPO) receptor, c-kit, CD34, CD105 (endoglin), CD31 (9PECAM-1), JAM-A, Tie-2, KDR (VEGF receptor 2) and a combination thereof. In one example, the one or more surface proteins comprise CD41. In another example, the one or more surface proteins comprise VLA-4 (CD49d).

According to the preparation method, the polymer may be poly(lactic-co-glycolic acid) (PLGA).

Where the active agent is hydrophobic, the preparation method may further comprise preparing the core from single-emulsion synthesis.

Where the active agent is hydrophilic, the preparation method may further comprise preparing the core from a double emulsion.

The active agent may be selected from the group consisting of an imaging agent, a therapeutic agent, and a combination thereof.

The imaging agent may be selected from the group consisting of fluorophores, MRI contrast agents, CT contrast agents, ultrasound contrast agents, and a combination thereof. The therapeutic agent may be a nucleic acid molecule selected from the group consisting of siRNA, miRNA, DNA, and a combination thereof. The DNA may be a single-stranded DNA. The therapeutic agent may be selected from the group consisting of chemotherapeutics, HSPC mobilizing agents, and a combination thereof. The therapeutic agent may be a chemotherapeutic.

The preparation method may further comprise mixing the active agent and the polymer to make the core. The preparation method may further comprise mixing the active agent, the polymer and an excipient to make the core.

Bio-nanoparticles prepared according to any preparation method of the present invention.

The bio-nanoparticles of the present invention may have an average diameter of 50-1000 nm. The biological membrane surrounding the core of the bio-nanoparticles may have a thickness of 7-10 nm. The bio-nanoparticles may bind HSPCs with a specificity greater than 90%. The bio-nanoparticles may be capable of entering HSPCs.

A composition for delivering an active agent into a hematopoietic stem & progenitor cell (HSPC) is provided. The composition comprises an effective amount of the bio-nanoparticles of the present invention. The composition may further comprise a carrier. The composition may further comprise a second active agent.

A method for delivering an active agent into hematopoietic stem & progenitor cells (HSPCs) is provided. The delivery method comprises introducing to the HSPCs bio-nanoparticles or a composition of the present invention such that the active agent is delivered into the HSPCs. The active agent may remain active in the HSPCs.

The hematopoietic stem and progenitor cells (HSPCs) may be from a first subject. The hematopoietic stem and progenitor cells (HSPCs) may be produced from an induced pluripotent stem cell (iPSC), cord blood stem cell, or embryonic stem cell.

The delivery method may further comprise administering the HSPCs having the active agent to a second subject. The second subject may have a disease or condition. The disease or condition may be selected from the group consisting of bone marrow failure disorder, leukemia, lymphoma, multiple myeloma, aplastic anemia, sickle cell disease, thalassemia, autoimmune disorders, HIV, multiple sclerosis, myeloproliferative disorder and myelodysplastic syndrome. In one embodiment, the disease or condition is cancer.

The delivery method may further comprise treating a disease or condition in the second subject. The disease or condition may be selected from the group consisting of bone marrow failure disorder, leukemia, lymphoma, multiple myeloma, aplastic anemia, sickle cell disease, thalassemia, autoimmune disorders, HIV, multiple sclerosis, myeloproliferative disorder and myelodysplastic syndrome. In one embodiment, the disease or condition is cancer.

The delivery method may further comprise preventing a disease or condition in the second subject. The disease or condition may be selected from the group consisting of bone marrow failure disorder, leukemia, lymphoma, multiple myeloma, aplastic anemia, sickle cell disease, thalassemia, autoimmune disorders, HIV, multiple sclerosis, myeloproliferative disorder and myelodysplastic syndrome. In one embodiment, the disease or condition is cancer.

A method for treating a disease or condition in a subject in need thereof is provided. The treatment method may comprise administering to the subject an effective amount of the bio-nanoparticles or the composition of the present invention. The disease or condition may be selected from the group consisting of bone marrow failure disorder, leukemia, lymphoma, multiple myeloma, aplastic anemia, sickle cell disease, thalassemia, autoimmune disorders, HIV, multiple sclerosis, myeloproliferative disorder, myelodysplastic syndrome, and other forms of cancer. In one embodiment, the disease or condition is cancer.

A method for preventing a disease or condition in a subject in need thereof is provided. The prevention method may comprise administering to the subject an effective amount of the bio-nanoparticles of the presentation invention. The disease or condition may be selected from the group consisting of bone marrow failure disorder, leukemia, lymphoma, multiple myeloma, aplastic anemia, sickle cell disease, thalassemia, autoimmune disorders, HIV, multiple sclerosis, myeloproliferative disorder, myelodysplastic syndrome, and other forms of cancer. In one embodiment, the disease or condition is cancer.

### BRIEF DESCRIPTION OF THE DRAWINGS

FIG. 1 illustrates one embodiment according to the present invention. Synthetic nanoparticles (NPs) composed of poly(lactic-co-glycolic acid (PLGA)) can be loaded with desired hydrophobic or hydrophilic cargo, for example, RNA depicted as the cargo here, and wrapped with biological membranes derived from the cytoplasmic membrane of megakaryocytes (Mks). The resultant Mk membrane-wrapped NPs (MkNPs), also called biomembrane-covered nanoparticles (BioNPs), can specifically bind and enter HSPCs to deliver their encapsulated cargo.

FIG. 2 shows characterization of MkNPs. (A) Transmission electron micrographs of bare PLGA NPs (“Bare NPs”), empty Mk membrane vesicles (“Empty Mk Membranes” or “Mk Membranes”) and Mk membrane-wrapped NPs (MkNPs or “Mk-Wrapped NPs”), which were prepared and placed on 400 nm copper grids and stained with uranyl acetate. The bare NPs showed a monodisperse spherical shape, while the empty Mk membranes appeared as hollow shells. The MkNPs, also known as BioNPs, exhibited a core/shell structure indicative of successful membrane wrapping or covering nanoparticles. (B) Mean intensity size diameter of bare NPs, Mk membrane vesicles, or MkNPs measured by nanoparticle tracking analysis. (C) Zeta potential of bare NPs, Mk membranes and MkNPs provides further confirmation of successful membrane wrapping in MkNPs. (D) Flow cytometry analysis of CD41 detected on whole Mk cells, Mk membranes and MkNPs. The percentage of the detected CD41 is similar for each sample, indicating that the membrane protein content was maintained during the membrane collection and nanoparticle cloaking process for making the MkNPs.

FIG. 3 shows purification of MkNPs. (A) Hydrodynamic diameter of bare NPs and MkNPs after being placed in water or phosphate buffered saline (PBS) for 1 hour. Bare NPs rapidly swell in PBS, while MkNPs maintained their size. This allows MkNP samples to be purified by placing samples of bare NPs and MkNPs in PBS to swell any bare NPs or unwrapped NPs, which can then be removed by filtration. (B) Morphology of synthesized MkNPs (top panels) and purified MkNPs (bottom panels) as visualized by transmission electron microscopy. (C) Size distribution curves of bare NPs, synthesized MkNPs, and purified MkNPs as determined by nanoparticle tracking analysis (NTA). Bare NPs with peak diameter of about 80 nm were wrapped with empty Mk membrane vesicles (MkMVs) approximately 150 nm in diameter by co-extruding them through a porous membrane. NTA analysis demonstrated that “synthesized MkNPs” contained fully wrapped MkNPs, excess bare NPs and empty MkMVs. These excess bare NPs and empty MkMVs could be removed by a combination of filtration and ultracentrifugation to produce purified MkNPs with a peak size centered at about 110 nm.

FIG. 4 shows reproducible MkNP synthesis. The bar graphs on the left side of each of (A), (B) and (C) show the intensity peak diameter and zeta potential measurements for three different batches of each of (A) bare NPs, (B) Mk membranes, and (C) Mk membrane-wrapped NPs (MkNPs). The light gray, black, and dark grey bars (from left to right) in each bar graph represent the three different batches, and demonstrate that the size and charge of bare NPs, Mk membranes, and membrane-wrapped MkNPs are consistent across batches. The four-panel transmission electron micrographs on the right side of each of (A), (B) and (C) are provided for four different synthesis batches for each of (A) bare NPs, (B) Mk membranes, and (C) MkNPs, further supporting that the synthesis of MkNPs is reliable. In top left panel of (C), bars are provided to indicate the thickness of the Mk membrane coating surrounding the PLGA NPs, which was determined to be 7-10 nm.

FIG. 5 shows internalization of MkNPs by HSPCs. (A) Confocal microscopy image of an HSPC interacting with MkNPs. The Mk membranes were labeled with PKH26 and the NPs were filled with DiD fluorophores. The HSPC nucleus is stained with DAPI. Both PKH26 and DiD signals are present in the HSPC, and co-localization of signals in the merged image indicates the MkNPs are intact following uptake by HSPCs. Scale bar, 10 μm. (B) MkNP uptake visualized in HSPCs after 24 hrs incubation using a 40× objective. Stills taken from Z-stack video in sequence are presented. The arrows point to representative MkNPs within the cells. Scale bar, 10 μm. (C) Fixed HSPCs with internalized MkNPs visualized under 60× magnification. HSPC membranes were stained with phalloidin and nuclei were stained with DAPI. MkNP membranes were labeled with PKH26 and they contained fluorescent DiD cargo. Still images taken from a Z-stack video in sequence are presented. The arrows point to representative internalized MkNPs as the HSPC nucleus comes into focus. Scale bar, 5 μm. All samples were observed using a Confocal LMS880 microscope.

FIG. 6 shows MkNPs in HSPC cytoplasm. HSPCs cultured with MkNPs were fixed and observed by super-resolution microscopy using a Zeiss Elyra PS 1 to visualize internalized MkNPs. HSPC membranes were stained with phalloidin and nuclei were stained with DAPI. MkNPs were stained with PKH26 membrane markers and loaded with DiD. Stills taken from a Z-stack video in sequence for two different cells (one cell on each row) are presented. The arrows point to internalized MkNPs when the nucleus comes into focus. Scale bars, 5 μm.

FIG. 7 shows that MkNPs preferentially target HSPCs versus alternative cell types. (A) Scheme of the co-culture setup to examine MkNP specificity for HSPCs versus other cell types. DID-loaded bare NPs or MkNPs were cultured with HSPCs, mesenchymal stem cells (MSCs), or human umbilical vein endothelial cells (HUVECs) in transwell inserts at various NP doses. Flow cytometry or microscopy were then performed to assess MkNP/cell interactions. (B) Flow cytometry analysis of MkNP uptake by HSPCs, HUVECs, or MSCs after different incubation periods based on DiD signal (indicative of particle delivery). Bare NPs exhibited equal uptake by all cell types (not shown), indicating lack of discrimination, whereas MkNPs exhibit preferential uptake by HSPCs versus non-targeted HUVECs or MSCs. (C) Confocal microscopy (Zeiss LSM880) images of HSPCs, HUVECs, and MSCs incubated with DiD-loaded MkNPs. The MKNP membranes were labeled with PKH26. The cell nuclei were labeled with DAPI and the actin cytoskeleton was labeled with Phalloidin. MkNPs are found within HSPCs, but not within non-targeted HUVECs or MSCs.

FIG. 8 shows characterization of MkNPs loaded with hydrophilic siRNA cargo. (A) Transmission electron micrographs of bare PLGA NPs loaded with siRNA (left panel), empty membrane vesicles derived from CHRF cells (which are an Mk-committed cell line) (center panel), and siRNA-loaded MkNPs prepared by wrapping CHRF membranes around siRNA-loaded PLGA NPs (right panel). The core/shell structure visible in the image in the right panel indicates successful membrane wrapping. (B) Mean diameter and (C) zeta potential of bare siRNA-loaded PLGA NPs, empty MkMVs, or membrane-wrapped siRNA-loaded MkNPs. The size and zeta potential increase observed for the MkNPs compared to bare NPs is indicative of membrane wrapping.

FIG. 9 shows that MkNP cargo remains functional upon delivery to targeted HSPCs. HSPCs were co-cultured with MkNPs containing siRNA targeting CD34 (a membrane marker of HSPCs) or containing negative control non-silencing siRNA (siNeg) for 24, 48, 72, or 96 hours, then flow cytometry was used to analyze CD34 expression by the HSPCs. Data shown is the deviation in CD34 expression relative to untreated HSPCs. MkNPs carrying siCD34 cargo significantly reduced CD34 expression in HSPCs versus MkNPs carrying siNeg. *p<0.05 (student's t-test).

### DETAILED DESCRIPTION OF THE INVENTION

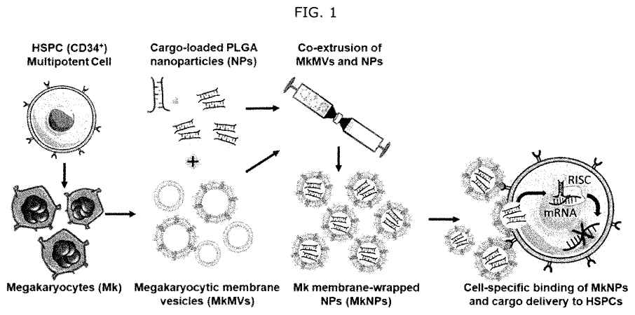

The present invention relates to highly reproducible biomembrane-covered nanoparticles, also known as bio-nanoparticles or BioNPs, carrying active agents and the use of such BioNPs for targeted delivery of the active agents into hematopoietic stem & progenitor cells (HSPCs). The invention is based on the surprising discovery by the inventors of a delivery system that can encapsulate and protect desired cargo, including hydrophobic molecules such as drugs and fluorophores, and hydrophilic cargo such as siRNA, miRNA and DNA, and specifically deliver the cargo to HSPCs in vitro or in vivo (FIG. 1). The inventors have synthesized BioNPs having PLGA NPs wrapped in Mk-derived membranes at high encapsulation efficiency and reproducibility. The BioNPs can bind and enter HSPCs to deliver various types of cargo with high specificity and controlled release of the cargo in the HSPCs.

BioNPs are a unique technology that can provide cargo delivery specifically to targeted HSPCs while avoiding non-targeted cells. Although NPs wrapped in membranes derived from red blood cells, platelets, or cancer cells have been previously described and utilized for hydrophobic drug delivery, no studies have reported the use of BioNPs wrapped in Mk-derived membranes for targeted drug delivery to HSPCs. Further, no studies have reported the delivery of hydrophilic cargo (such as nucleic acids) to HSPCs with BioNPs. The inventors has unexpectedly discovered that: (i) BioNPs can be synthesized using Mk-derived membranes to surround PLGA NPs, (ii) BioNPs can be loaded with either hydrophobic or hydrophilic cargo, (iii) BioNP synthesis is reproducible; (iv) BioNPs can bind and enter HSPCs; (v) BioNPs can deliver cargo into HSPCs, and this cargo remains functional inside the cells.

The invention provides a bio-nanoparticle (BioNP) for delivering an active agent into a hematopoietic stem & progenitor cell (HSPC). The BioNP comprises a core and a biological membrane covering the core. The core comprises the active agent and a polymer. The biological membrane comprises a phospholipid bilayer and one or more surface proteins of a megakaryocyte (Mk). The active agent may remain active after being delivered into the HSPC. The biological membrane may be adhered to the core by an electrostatic interaction.

The BioNPs of the present invention may have an average diameter of 1-2000 nm, 10-1000 nm, 50-1000 nm, 50-500 nm, 50-200 nm, 75-150 nm, 90-130 nm, 100-120 nm or 105-115 nm.

The BioNPs of the present invention may bind HSPCs. The term “specificity” as used herein refers to the percentage of cells, for example, HSPCs, red blood cells, platelets, cancer cells, mesenchymal stem cells (MSCs), or human umbilical vein endothelial cells (HUVECs), that are bound by the BioNPs after the cells are incubated with an excess amount of the BioNPs. The BioNPs may bind HSPCs with a specificity of at least 50%, 60%, 70%, 80%, 90%, 95% or 99%. The binding specificity of the BioNPs for HSPCs may be at least 50%, 60%, 70%, 80%, 90%, 100%, 200%, 300%, 400% or 500% higher than that for red blood cells, platelets, cancer cells, MSCs or HUVECs.

The BioNPs may be capable of entering HSPCs. The release of the active agent from the BioNPs in the HSPCs may be controlled by the ingredients in the BioNPs, for example, the polymer. After the HSPCs are incubated with an excess amount of the BioNPs, at least 5%, 10%, 15%, 20%, 25%, 30%, 35%, 40%, 45%, 50%, 60%, 70%, 80%, 90% or 95%, or about 5-95%, 10-90%, 20-50% or 20-30% of the active agent may be released from the BioNPs in the HSPCs within, for example, 1, 2, 3, 4, 5, 6, 7, 8, 9, 10, 11, 12, 18, 24, 36, 48, 72, 96, or 120 hours.

The active agent of the present invention may be any agent having an activity and remain active after being delivered into the HSPC according to the present invention. At least about 50%, 60%, 70%, 80%, 90% or 95% of the activity of the active agent remains after the active agent is delivered into the HSPC.

The active agent may be a compound, a biological molecule or a combination thereof. The active agent may be an imaging agent, a therapeutic agent, or a combination thereof. The imaging agent may be selected from the group consisting of fluorophores, MRI contrast agents, CT contrast agents, ultrasound contrast agents, and combinations thereof. The therapeutic agent may be a nucleic acid molecule selected from the group consisting of siRNA, miRNA, DNA, and a combination thereof. The DNA may be a single-stranded DNA. The therapeutic agent may be a chemotherapeutic, a HSPC mobilizing agent and a combination thereof. An HSPC mobilizing agent is a drug that is used to stimulate the movement of HSPCs from a patient's bone marrow into their peripheral blood. Examples of the HSPC mobilizing agents include granulocyte colony stimulating factor, granulocyte/macrophage colony stimulating factor, ADM3100, or a combination thereof. In one embodiment, the therapeutic agent is a chemotherapeutic.

The polymer may be any biodegradable polymer. Examples of the polymer include poly(lactic-co-glycolic acid) (PLGA).

The core may be prepared from a single-emulsion or double-emulsion depending on the nature of the active agent. For a hydrophobic active agent, the core may be prepared from a single-emulsion. For a hydrophilic active agent, the core may be prepared from a double-emulsion. For example, PLGA NPs containing hydrophobic cargo (e.g., fluorophores, drugs) can be prepared by single emulsion solvent evaporation, which involves dissolving PLGA in acetone along with the desired hydrophobic molecules and then adding this solution dropwise to water at a specified ratio. Alternatively, PLGA NPs containing hydrophilic cargo (e.g., siRNA, miRNA, DNA) can be prepared by a double-emulsion solvent evaporation method. In this method, the hydrophilic cargo and any desired excipients are dissolved in water, then added dropwise to a solution of PLGA in acetone. This first emulsion is then added to water at a desired ratio to produce PLGA NPs. Once PLGA NPs containing hydrophobic or hydrophilic cargo are synthesized, they are stirred for several hours to allow the acetone solvent to evaporate, and then they are centrifuged to remove any non-encapsulated cargo and collect the desired end product. Notably, the diameter of PLGA NPs containing hydrophobic or hydrophilic cargo can be adjusted across a broad range (e.g., spanning 50 nm to 1000 nm) by adjusting the ratio of PLGA to acetone, the volume of cargo added, the rate of mixing, and other features. Similarly, the cargo loading and release profile can be adjusted by tailoring the ratio of lactic to glycolic acids, the inherent viscosity of the polymer, the amount and type of cargo added to the synthesis solution, and the type and amount of excipients (e.g., polyvinyl alcohol, poly-1-lysine, etc.) loaded in the PLGA NPs.

The core may be a nanoparticle. The core may have an average diameter of 50-1000 nm, 50-500 nm, 50-200 nm, 50-120 nm, 60-100 nm, 70-90 nm or 75-85 nm.

The core may further comprise an excipient. Suitable excipients include poly-L-arginine, poly-L-lysine, polyethylenimine, and polyvinyl alcohol.

The term “biological membrane” used herein refers to a plasma membrane having a phospholipid bilayer and at least one surface protein of a megakaryocyte (Mk). The Mk surface protein may be any protein on the surface of an Mk, for example, a receptor. Examples of the Mk surface proteins include CD62P, VLA-4 (CD49d), CD41, CD150, CXCR4, thrombopoietin (TPO) receptor, c-kit, CD34, CD105 (endoglin), CD31 (9PECAM-1), JAM-A, Tie-2 and KDR (VEGF receptor 2). The Mk surface protein may not be present on the surface of another cell such as a red blood cell, platelet, cancer cell, MSC or HUVEC. In one embodiment, the Mk surface protein is CD41. In another embodiment, the Mk surface protein is VLA-4 (CD49d).

The biological membrane to be used for wrapping, encapsulating or covering the core may have an average diameter of 50-1000 nm, 100-1000 nm, 125-250 nm or 150-200 nm. The biological membrane in the BioNPs may have a thickness of 7-10 nm, 5-10 nm, 5-15 nm, or 10-15 nm.

The biological membrane may be prepared from a cell, directly or indirectly. For example, the biological membrane may be prepared directly from a megakaryocyte (Mk), a megakaryocytic microparticle (MkMP) or a megakaryocytic extracellular vesicle. The megakaryocyte (Mk), a megakaryocytic microparticle (MkMP) or a megakaryocytic extracellular vesicle may be prepared or differentiated from a human megakaryocyte cell line, or from primary Mk cells. The term “megakaryocytic microparticle (MkMP)” used herein refers to extracellular vesicles budding off the cytoplasmic membrane of Mk cells and may have an average diameter of 50-1000 nm, 100-1000 nm, 125-250 nm or 150-200 nm. The term “megakaryocytic extracellular vesicle” used herein refers to lipid bilayer-delimited particles that are naturally released from Mk cells and do not possess the ability to replicate. The megakaryocytic extracellular vesicle may have an average diameter of 50-1000 nm, 100-1000 nm, 125-250 nm or 150-200 nm. The MkMPs and the megakaryocytic extracellular vesicle share the same membrane structure with the Mk plasma membrane, including the same phospholipid bilayer and surface proteins of the Mk cells. Thus, the biological membrane derived from Mk cells, MkMPs or megakaryocytic extracellular vesicles contain the same phospholipids and corresponding surface proteins of the Mk cells, which are critical to their biological function (i.e., their HSPC-specific targeting capabilities). As shown in FIG. 1, the bio-nanoparticles (BioNPs) produced by wrapping Mk-derived biological membranes around bare nanoparticles (NPs), for example, PLGA NPs, would maintain the unique HSPC-target recognition abilities of the source Mk cells or MkMPs, enhancing cargo delivery to HSPCs in vitro or in vivo.

In one embodiment, Mk cells are used to extract Mk-membrane vesicles (MkMVs) for wrapping the NPs. Briefly, whole Mk cells are collected, placed in a hypotonic lysis buffer and homogenized to disrupt the cells. A multi-step centrifugation process is then performed to remove intracellular components of the Mk cells and collect the plasma membrane pellet, which contains the MkMVs. The MkMVs are then extruded through a porous membrane to produce vesicles of the desired size. The MkMVs could also be produced from Mk cells by free-thaw or electroporation methods.

The BioNPs of the present invention offer several advantages as a platform to address the challenge of delivering active agents to HSPCs, for example, when PLGA is used as the polymer in the core. This includes: (i) PLGA is a non-toxic, bio-degradable polymer that has been cleared for use in drug delivery by the Food & Drug Administration (FDA); (ii) PLGA NPs have a large carrying capacity and can be loaded with hydrophobic or hydrophilic cargo (indeed, it has been shown that PLGA NPs can be loaded with siRNA, DNA, chemotherapeutics, fluorophores, and more); (iii) PLGA NPs have tunable physicochemical properties and can also be loaded with excipients to optimize cargo loading and release profiles; (iv) BioNPs wrapped in Mk-derived membranes can specifically bind and enter HSPCs while exhibiting minimal uptake by non-targeted cells; and (v) BioNPs can protect their cargo, which maintains its function upon delivery to the targeted cells.

A method for preparing a bio-nanoparticle (BioNP) for delivering an active agent into a hematopoietic stem & progenitor cell (HSPC) is provided. The preparation method comprises coating a core with a biological membrane. The core comprises an active agent and a polymer.

According to the preparation method of the present invention, the core may be a nanoparticle. The core may have an average diameter of 50-1000 nm, 50-500 nm, 50-200 nm, 50-120 nm, 60-100 nm, 70-90 nm or 75-85 nm. The core may further comprise an excipient. Suitable excipients include poly-L-arginine, polyvinyl alcohol, poly-L-lysine, or polyethylenimine.

According to the preparation method of the present invention, the active agent may be any agent having a biological activity. The active agent may be a compound, a biological molecule or a combination thereof. The active agent may be an imaging agent, a therapeutic agent, or a combination thereof. The imaging agent may be selected from the group consisting of fluorophores, MRI contrast agents, CT contrast agents, ultrasound contrast agents, and combinations thereof. The therapeutic agent may be a nucleic acid molecule selected from the group consisting of siRNA, miRNA, DNA, and a combination thereof. The DNA may be a single-stranded DNA. The therapeutic agent may be a chemotherapeutic, a HSPC mobilizing agent and a combination thereof. An HSPC mobilizing agent is a drug that is used to stimulate the movement of HSPCs from a patient's bone marrow into their peripheral blood. Examples of the HSPC mobilizing agent include granulocyte colony stimulating factor, granulocyte/macrophage colony stimulating factor, ADM3100, or a combination thereof. In one embodiment, the therapeutic agent is a chemotherapeutic.

According to the preparation method of the present invention, the polymer may be any biodegradable polymer. Examples of the polymer include poly(lactic-co-glycolic acid) (PLGA).

The preparation method may further comprise preparing the core. The core may be prepared from a single-emulsion or double-emulsion depending on the nature of the active agent. For a hydrophobic active agent, the preparation method may further comprise preparing the core from a single-emulsion. For a hydrophilic active agent, the preparation method may further comprise preparing the core from a double-emulsion.

The preparation method may further comprise mixing the active agent and the polymer to make the core. The preparation method may further comprise mixing the active agent, the polymer and the excipient to make the core.

According to the preparation method of the present invention, the biological membrane comprises a phospholipid bilayer and one or more surface proteins of a megakaryocyte (Mk). The Mk surface protein may be any protein on the surface of an

Mk, for example, a receptor. Examples of the Mk surface proteins include CD62P, VLA-4 (CD49d), CD41, CD150, CXCR4, thrombopoietin (TPO) receptor, c-kit, CD34, CD105 (endoglin), CD31 (9PECAM-1), JAM-A, Tie-2 and KDR (VEGF receptor 2). The Mk surface protein may not be present on the surface of another cell such as a red blood cell, platelet, cancer cell, MSC or HUVEC. In one embodiment, the Mk surface protein is CD41. In another embodiment, the Mk surface protein is VLA-4 (CD49d).

The term “efficiency of encapsulation” or “encapsulation efficiency” as used herein refers to the weight percentage of the core is encapsulated, covered or wrapped by the biological membrane after mixing the core with the biological membrane. The encapsulation efficiency may be at least 80%, 90%, 95%, 99% or 99.9%.

The encapsulation efficiency may be improved by adjusting the weight ratio of the core to the biological membrane. Excess amount of the biological membrane may improve encapsulation efficiency. For example, the weight ratio of the biological membrane to the core may be at least 1:1, 1.5:1, 2:1, 2.5:1, 3:1, 5:1 or 10:1.

The encapsulation efficiency may be improved by mixing the core and the biological membrane having a desired diameter, which may be 0.01-1 μm, 0.1-0.9 μm, 0.2-0.8 μm, 0.3-0.9 μm or 0.2-0.9 μm. For example, the core and the biological membrane may be co-extruded through a porous membrane having the same desired diameter of, for example, 0.01-1 μm, 0.1-0.9 μm, 0.2-0.8 μm, 0.3-0.9 μm or 0.2-0.9 μm.

In one embodiment, PLGA NPs and MkMVs of the desired diameter are produced, and co-extruded through a porous membrane (e.g., 0.2-0.8 μm) to produce membrane-wrapped BioNPs. Successful membrane wrapping may be facilitated by the asymmetric charge of the cell membrane, which would cause MkMVs to orient properly (i.e., right side out) on the PLGA NPs owing to charge repulsion between the negative extracellular membrane components and the negative surface of the PLGA NPs. An excess amount of MkMVs may be used to wrap PLGA NPs at a weight ratio of MkMVs to PLGA NPs of 1:1, 2:1, or higher, to ensure complete membrane wrapping.

The preparation method may further comprise preparing the biological membrane from a megakaryocyte (Mk), megakaryocytic microparticle or megakaryocytic extracellular vesicle. The preparation method may further comprise preparing the megakaryocyte (Mk), megakaryocytic microparticle or megakaryocytic extracellular vesicle from a hematopoietic stem & progenitor cell (HSPC) or a human megakaryocyte cell line. The biological membrane used to wrap, encapsulate or cover the core may have an average diameter of 50-1000 nm, 100-1000 nm, 125-250 nm or 150-200 nm. The biological membrane in the BioNPs may have a thickness of 7-10 nm, 5-10 nm, 5-15 nm, or 10-15 nm.

The preparation method may further comprise preparing the biological membrane from a megakaryocyte (Mk) after one or more components of the Mk are removed from the Mk. The one or more components may be selected from the group consisting of cytosolic, nuclear and mitochondrial components. Examples of cytosolic components of the Mk include the cytosol and organelles. The nuclear components of the Mk may be DNA, histones, chromosomes, nuclear envelope, and the nuclear matrix. Exemplary mitochondrial components of the Mk include mitochondrial DNA, mitochondrial membranes, and the mitochondrial matrix. In one embodiment, the biological membrane does not contain cytosolic components, nuclear components, or mitochondrial components.

The preparation method may further comprise adhering the biological membrane to the core by an electrostatic interaction. For example, negatively charged nanoparticles may repel negatively charged components of the outer cellular membrane, resulting in right-side-out orientation of the membrane on the nanoparticle core. The electrostatic interaction between the biological membrane and the core could be determined by conventional technique known in the art, for example, zeta potential analysis.

For each preparation method of the present invention, BioNPs prepared according to the preparation method are provided. The BioNPs may have an average diameter of 1-2000 nm, 10-1000 nm, 50-1000 nm, 50-500 nm, 50-200 nm, 75-150 nm, 90-130 nm, 100-120 nm or 105-115 nm. The BioNPs may bind HSPCs with a specificity of, for example, at least 50%, 60%, 70%, 80%, 90%, 95% or 99%. The BioNPs may be capable of entering HSPCs. After the HSPCs are incubated with an excess amount of the BioNPs, at least 5%, 10%, 15%, 20%, 25%, 30%, 35%, 40%, 45%, 50%, 60%, 70%, 80%, 90% or 95%, or about 5-95%, 10-90%, 20-50% or 20-30% of the active agent may be released from the BioNPs in the HSPCs within, for example, 1, 2, 3, 4, 5, 6, 7, 8, 9, 10, 11, 12, 18, 24, 36, 48, 72, 96, or 120 hours.

A composition for delivering an active agent into HSPCs is provided. The composition comprises an effective amount of the BioNPs of the present invention. The BioNPs comprise the active agent. The composition may comprise the BioNPs at a concentration of at least 1,000, 5,000, 10,000, 50,000 or 100,000 per HSPCs. The composition may further comprise a carrier. Suitable carriers include larger nanoparticles or microparticles, hydrogels, or polymer. The composition further comprise a second active agent. The second active agent may be selected from the group consisting of chemotherapeutic agents, nucleic acids, HSPC mobilizing agents and imaging agents. The second active agent may be selected from the group consisting of chemotherapeutic agents, nucleic acids and imaging agents.

A method for delivering an active agent into HSPCs is provided. The delivery method comprises introducing an effective amount of the BioNPs of the present invention to the HPSCs. The BioNPs comprise the active agent. As a result, the active agent is delivered into the HSPCs and the active agent remains active in the HSPC.

According to the delivery method, the HSPCs may be from a first subject, for example, a mammal, preferably a human. The HSPCs may be produced from an induced pluripotent stem cell (iPSC), cord blood stem cell, or embryonic stem cell.

The delivery method may further comprise administering the HSPC having the active agent to a second subject. The second subject may be the same as the first subject. The delivery method may further comprise treating or preventing a disease or condition in the second subject. The disease or condition may be selected from the group consisting of bone marrow failure disorder, leukemia, lymphoma, multiple myeloma, aplastic anemia, sickle cell disease, thalassemia, autoimmune disorders, HIV, multiple sclerosis, myeloproliferative disorder and myelodysplastic syndrome. For example, the disease or condition is cancer.

A method for treating a disease or condition in a subject in need thereof is provided. The treatment method comprises administering to the subject an effective amount of the BioNPs of the present invention. The disease or condition may be selected from the group consisting of bone marrow failure disorder, leukemia, lymphoma, multiple myeloma, aplastic anemia, sickle cell disease, thalassemia, autoimmune disorders, HIV, multiple sclerosis, myeloproliferative disorder, myelodysplastic syndrome, and other forms of cancer. In one embodiment, the disease or condition is cancer.

A method for preventing a disease or condition in a subject in need thereof is provided. The prevention method comprises administering to the subject an effective amount of the BioNPs of the present invention. The disease or condition may be selected from the group consisting of bone marrow failure disorder, leukemia, lymphoma, multiple myeloma, aplastic anemia, sickle cell disease, thalassemia, autoimmune disorders, HIV, multiple sclerosis, myeloproliferative disorder, myelodysplastic syndrome, and other forms of cancer. In one embodiment, the disease or condition is cancer.

### EXAMPLES 1-4: SYNTHESIS AND CHARACTERIZATION OF BIONPS CONTAINING HYDROPHOBIC CARGO

To demonstrate the synthesis of BioNPs containing hydrophobic molecules, we used DiD fluorophores as model cargo, as this allows visualization of cargo delivery to HSPCs by fluorescence microscopy. In short, we synthesized PLGA NPs encapsulating DiD by the method described above method by dissolving 50:50 PLGA with an inherent viscosity of 0.67 dL/g in acetone along with DiD fluorophores and adding this mixture dropwise to water in a 1:3 ratio. We have adjusted the concentration of PLGA used in this synthesis from 1 to 4 mg/mL, resulting in particles ranging from 50 to 120 nm diameter. Likewise, we have used the above lysis and homogenization method to produce MkMVs approximately 150 nm in diameter, and we have co-extruded these MkMVs with DiD-loaded PLGA NPs to produce Mk membrane-wrapped BioNPs. The resultant BioNPs were characterized by several techniques, summarized below.

### Example 1: BioNPs are Successfully Wrapped with Mk-Derived Membranes

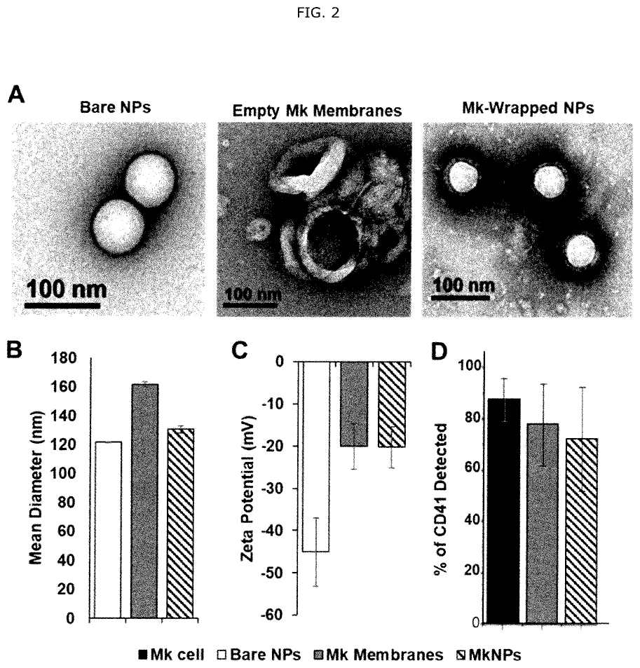

The successful production of BioNPs was confirmed by using transmission electron microscopy (TEM) of uranyl-acetate stained samples to visualize unwrapped (bare) PLGA NPs, empty MkMVs, and Mk membrane-wrapped BioNPs (FIG. 2A). As seen in these images, bare PLGA NPs have a homogenous spherical shape and MkMVs appear as hollow shells. By comparison, Mk membrane-wrapped BioNPs have core/shell structure indicative of PLGA NPs (brighter interior) surrounded by Mk-derived biological membranes (darker exterior).

The hydrodynamic diameter and zeta potential of bare NPs, MkMVs, and BioNPs were also measured to corroborate the TEM findings and confirm successful membrane wrapping. As shown in FIG. 2B, BioNPs are slightly larger than bare PLGA NPs, but smaller than empty MkMVs (which typically have a mean diameter ranging from 140-160 nm). In general, we have found by TEM and nanoparticle tracking analysis (NTA) that BioNPs are 10-20 nm diameter larger than bare PLGA NPs, which corresponds to the 7-10 nm thickness of the membranes. FIG. 2C shows the zeta potential (i.e., surface charge) of bare NPs, MkMVs, and BioNPs. Bare NPs typically have a charge of −40 to −60 mV, which increases to approximately −15 to −30 mV upon membrane wrapping as BioNPs take on the charge of the membrane vesicles.

### Example 2: Bionps Maintain the Membrane Composition of Their Source Cells

For BioNPs to maintain the unique HSPC-specific targeting capabilities of Mk cells and MkMPs, they must retain the characteristic membrane proteins. To confirm membrane composition is preserved after wrapping, we synthesized BioNPs as above, and then incubated the samples with a solution of 1-μm streptavidin beads decorated with antibodies against CD41, a surface marker of Mks. The antibodies on the beads can bind CD41 found on BioNPs, whole Mk cells, and empty Mk membrane vesicles. The samples can then be incubated with FITC-labeled anti-CD41 antibodies and analyzed by flow cytometry to determine the relative amount of CD41 present in each group. As shown in FIG. 2D, using this technique we determined that the fraction of streptavidin beads exhibiting positive CD41 signal in the case of whole Mk cells was approximately 85%, which reduced to approximately 75% for empty MkMVs and fully wrapped BioNPs. This indicates that CD41 levels are primarily maintained during membrane wrapping, which is imperative for BioNPs to exhibit HSPC-specific binding.

### Example 3: BioNPs can be Purified to Eliminate Excess Membrane Vesicles or Bare NPs

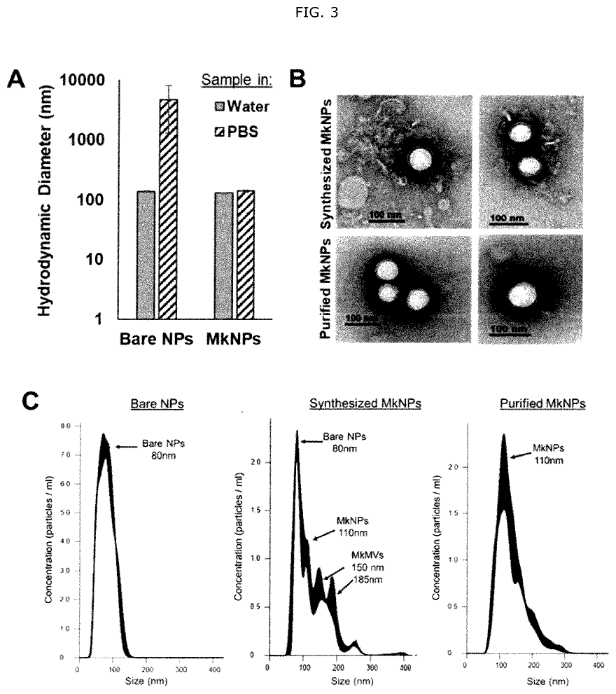

Nanoparticle tracking analysis (NTA) is an invaluable tool to analyze BioNPs, as it has the sensitivity necessary to distinguish bare NPs from empty MkMVs and fully wrapped BioNPs. As shown in FIG. 3, without additional purification, BioNPs prepared by single emulsion synthesis can contain not just fully wrapped NPs (indicated by a peak at 110 nm), but also bare NPs (indicated by a peak at 80 nm) and excess empty Mk membrane vesicles (peaks at 150 and 180 nm). We developed a method to purify fully wrapped BioNPs from bare NPs and empty MkMVs. In this method, “as-synthesized” BioNPs are suspended in phosphate buffered saline overnight, causing bare NPs to swell. The swollen bare NPs can then be removed by filtration, and the sample centrifuged to collect Mk membrane-wrapped BioNPs and remove excess MkMVs. The graph in the right of FIG. 3 shows the size of “purified” BioNPs as determined by NTA, with a single peak centered at 110 nm. This data confirms that BioNPs can be purified from starting products, which is imperative to ensure proper characterization and dosing in in vitro or in vivo studies.

### Example 4: BioNP Synthesis is Reproducible

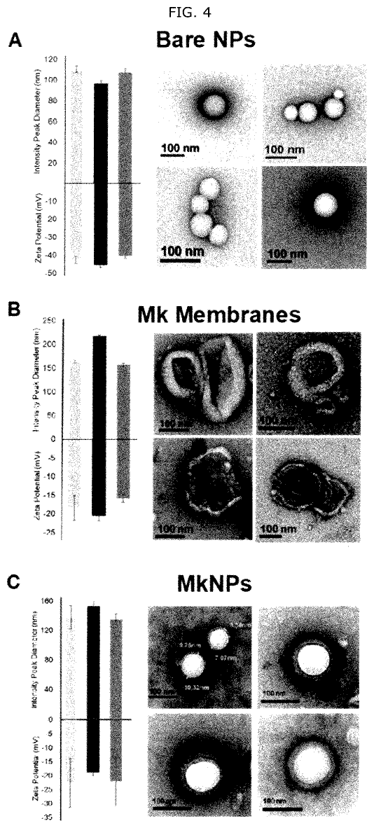

For BioNPs to be commercially relevant, it is important that their synthesis is reproducible. We have synthesized multiple batches of BioNPs using the methods described above, and characterized them by NTA, zeta potential measurements, and TEM. FIG. 4A shows the diameter and zeta potential of three different bare NP batches, as well as TEM images of bare NPs from four different batches. Critically, the size, charge, and structure of these particles are consistent from batch-to-batch. Similar data are provided for empty Mk membrane vesicles in FIG. 4B, and for fully wrapped BioNPs in FIG. 4C. These data confirm that BioNP synthesis is reproducible at the scale examined here.

### EXAMPLES 5-6: BIONPS PREFERENTIALLY INTERACT WITH HSPCS BUT NOT NON-TARGETED CELLS IN VITRO

The following examples provide evidence that BioNPs prepared as described above and loaded with DiD cargo can be internalized by HSPCs, while exhibiting minimal uptake by non-targeted cells. In contrast, bare NPs exhibit equivalent uptake by all cell types investigated. This demonstrates the advantage of wrapping NPs with Mk-derived membranes to facilitate HSPC-specific binding and uptake.

### Example 5: HSPCs Internalize BioNPs within 24 Hours

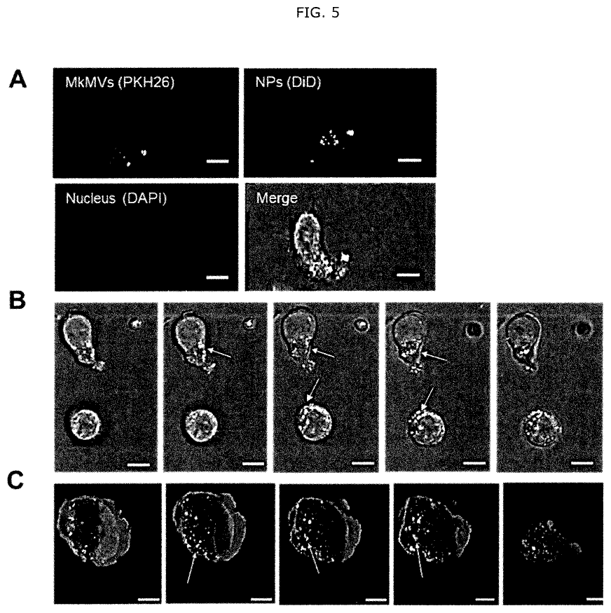

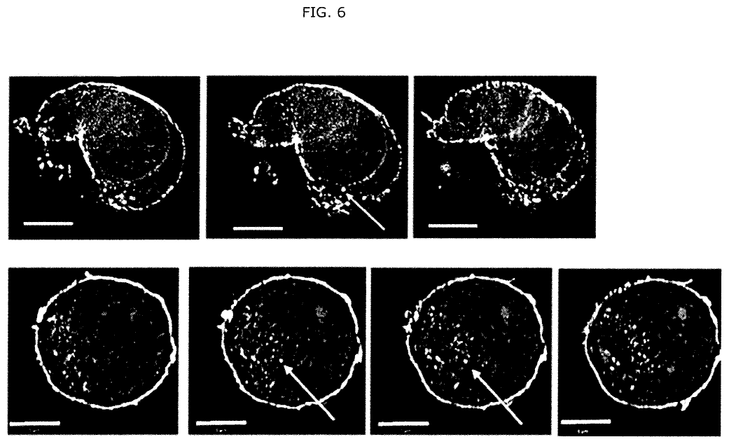

To substantiate that BioNPs can bind and enter HSPCs, as previously observed for MkMPs, we performed in vitro studies to assess the interaction between BioNPs and HSPCs (FIGS. 5 and 6). In these experiments, BioNPs were loaded with DiD fluorophores (ex 644 nm/em 665 nm) and their membranes labeled with PKH26 (ex 551 nm/em 567 nm) to enable visualization. After incubating the BioNPs with HSPCs for 24 hours, the cells were stained to visualize nuclei with DAPI and actin with Phalloidin. Confocal microscopy confirmed that BioNPs are internalized by HSPCs within this 24-hour incubation period (FIGS. 5 and 6). Both the PKH26 labels and DiD cargo are observed in the cytoplasm of the HSPCs when the nucleus is in focus, confirming the particles are not just bound to the cell exterior, but also internalized and that they remain intact inside the cell. These experiments have been repeated multiple times, and consistently demonstrate that BioNPs exhibit the ability to bind and enter HSPCs.

### Example 6: BioNPs Exhibit Minimal Binding to Non-Targeted Cells

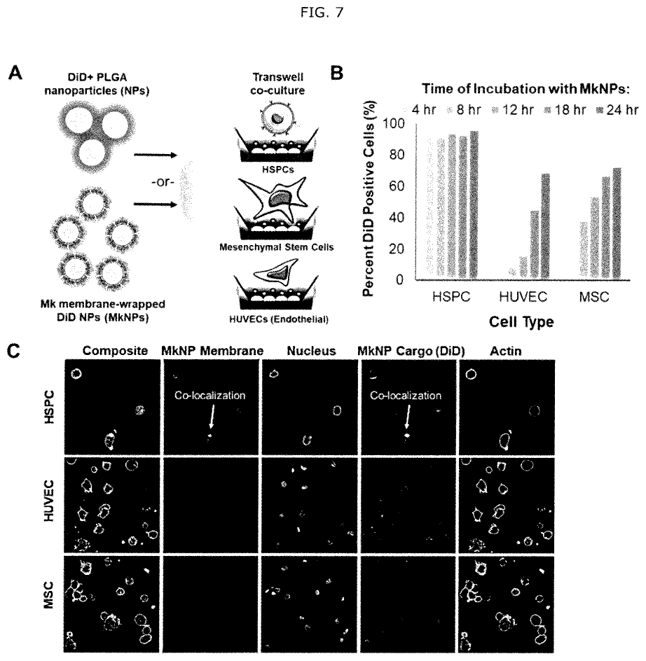

To confirm that BioNPs are specific for HSPCs versus non-targeted cell types, we performed in vitro studies wherein DiD-loaded BioNPs were incubated with HSPCs or with non-targeted mesenchymal stem cells (MSCs) or human umbilical vein endothelial cells (HUVECs) for time periods ranging from 4 hrs to 24 hrs (FIG. 7A). Each of the cell types were also incubated with DiD-loaded bare NPs, which should not exhibit preferential uptake by any particular cell type. Confirming this hypothesis, flow cytometry analysis of DiD signal in HSPCs, HUVECs, and MSCs showed that bare NPs were taken up equally by all three cell types (not shown). This demonstrates that bare NPs lack targeting specificity. By comparison, BioNPs exhibited higher uptake by HSPCs than HUVECs or MSCs at all time points studied (FIG. 7B). More specifically, >90% of HSPCs were positive for DiD signal indicate of BioNP uptake, while much fewer HUVECs or MSCs were positive for DiD (FIG. 7B). These data were corroborated by confocal microscopy studies, which showed that BioNPs were preferentially internalized by HSPCs, while exhibiting minimal uptake by non-targeted MSCs and HUVECs (FIG. 7C). Together, these findings confirm that cloaking PLGA NPs with Mk-derived membranes imparts them with unique HSPC-specific targeting capabilities.

### EXAMPLES 7-8: BIONPS CAN BE ENGINEERED TO DELIVER HYDROPHILIC CARGO TO HSPCS

The above examples illustrate that BioNPs can be loaded with hydrophobic cargo and deliver this cargo specifically to HSPCs. Below, we show that BioNPs can also be loaded with hydrophilic entities using siRNA targeting CD34 (a surface marker of HSPCs) as a model cargo. Further, we demonstrate that this cargo retains its function by showing BioNPs loaded with siCD34 can facilitate CD34 silencing in targeted HSPCs.

### Example 7: Characterization of siRNA-Loaded BioNPs

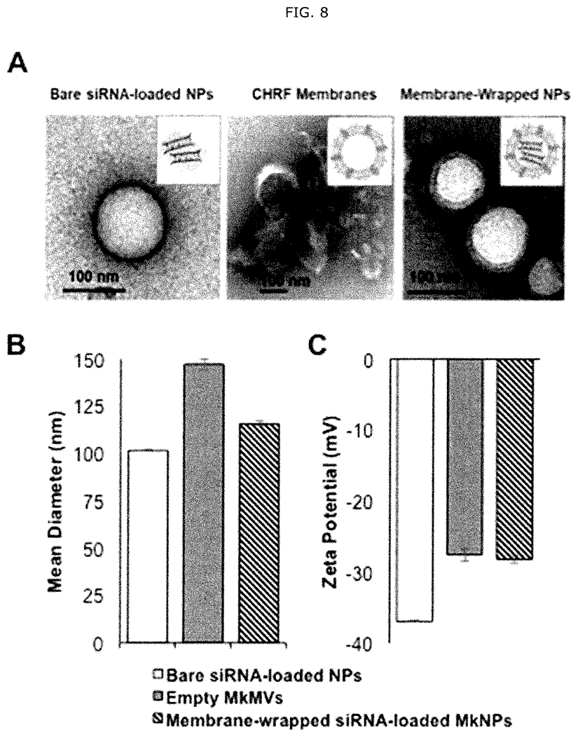

We have synthesized BioNPs loaded with siRNA (with amounts loaded ranging from 0.4 to 40 nmoles) by adapting a previously established double emulsion procedure (Pantazis, P., et al., Preparation of siRNA-encapsulated PLGA nanoparticles for sustained release of siRNA and evaluation of encapsulation efficiency. Methods Mol Biol, 2012. 906: p. 311-9), as described above. We characterized the size, zeta potential, and structure of siRNA-loaded BioNPs to confirm successful membrane wrapping. The TEM images presented in FIG. 8A show that siRNA-loaded BioNPs have the core/shell structure characteristic of PLGA NPs wrapped with Mk-derived membranes. Successful wrapping is further evidenced by size analysis data (FIG. 8B), which show that siRNA-loaded BioNPs are 10-20 nm larger than unwrapped siRNA-loaded NPs. Finally, siRNA-loaded BioNPs have a zeta potential matched to that of their source membranes (FIG. 8C), similar to what we observed for BioNPs loaded with DiD cargo. Together, these analyses confirm that BioNPs can be prepared to encapsulate hydrophilic cargo such as siRNA.

### Example 8: BioNPs Encapsulating siRNA can Silence Gene Expression in HSPCs In Vitro

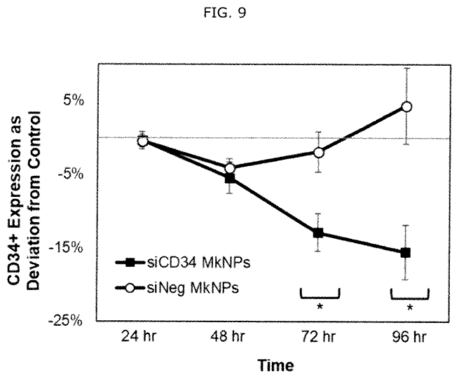

To demonstrate that the cargo loaded in BioNPs remains functional, we evaluated the ability of BioNPs carrying siRNA to silence CD34 expression in HSPCs. HSPCs were incubated with BioNPs carrying siCD34 or negative control siRNA for up to four days. After 24, 48, 72, or 96 hours, the samples were incubated with fluorophore-labeled anti-CD34 antibodies to bind any CD34 molecules still expressed on the HSPC surface, and then flow cytometry was performed. When CD34 is silenced, the signal observed in flow cytometry is reduced, enabling quantitative analysis of gene silencing. As shown in FIG. 9, CD34 was suppressed when HSPCs were exposed to BioNPs carrying siCD34, but not in the presence of BioNPs loaded with control siRNA. This finding confirms that BioNPs can deliver functional cargo into HSPCs to elicit desired effects. The proof-of-concept illustrated here with siCD34 opens the door to delivery of other functional cargo (e.g., siRNAs, DNAs, miRNAs, drugs, etc.) in the future.

All documents, books, manuals, papers, patents, published patent applications, guides, abstracts, and/or other references cited herein are incorporated by reference in their entirety. Other embodiments of the invention will be apparent to those skilled in the art from consideration of the specification and practice of the invention disclosed herein. It is intended that the specification and examples be considered as exemplary only, with the true scope and spirit of the invention being indicated by the following claims.

What is claimed:

1. A bio-nanoparticle for delivering an active agent into a hematopoietic stem & progenitor cell (HSPC), comprising a core and a biological membrane covering the core, wherein the core comprises the active agent and a polymer, wherein the biological membrane comprises a phospholipid bilayer and one or more surface proteins of a megakaryocyte (Mk), and wherein the active agent remains active after being delivered into the HSPC.

2. The bio-nanoparticle of claim 1, wherein the biological membrane is adhered to the core by an electrostatic interaction.

3. The bio-nanoparticle of claim 1, wherein the biological membrane is prepared from a megakaryocyte (Mk), megakaryocytic microparticle (MkMP) or megakaryocytic extracellular vesicle.

4. The bio-nanoparticle of claim 3, wherein the megakaryocyte (Mk), megakaryocytic microparticle or megakaryocytic extracellular vesicle is prepared from a hematopoietic stem & progenitor cell (HSPC) or a human megakaryocyte cell line.

5. The bio-nanoparticle of claim 3, wherein the biological membrane is prepared from a megakaryocyte (Mk) and the bio-nanoparticle lacks a cytosolic, nuclear or mitochondrial component of the Mk.

6. The bio-nanoparticle of claim 1, wherein the one or more surface proteins are selected from the group consisting of CD62P, VLA-4 (CD49d), CD41, CD150, CXCR4, thrombopoietin (TPO) receptor, c-kit, CD34, CD105 (endoglin), CD31 (9PECAM-1), JAM-A, Tie-2, KDR (VEGF receptor 2) and a combination thereof.

7. The bio-nanoparticle of claim 1, wherein the polymer is poly(lactic-co-glycolic acid) (PLGA).

8. The bio-nanoparticle of claim 1, wherein the active agent is hydrophobic and the core is prepared from a single-emulsion or double-emulsion.

9. The bio-nanoparticle of claim 1, wherein the active agent is selected from the group consisting of an imaging agent, a therapeutic agent, and a combination thereof, wherein the imaging agent is selected from the group consisting of fluorophores, MRI contrast agents, CT contrast agents, ultrasound contrast agents, and combinations thereof, wherein the therapeutic agent is a nucleic acid molecule selected from the group consisting of siRNA, miRNA, DNA, and a combination thereof, wherein the DNA is a single-stranded DNA, and wherein the therapeutic agent is selected from the group consisting of chemotherapeutics, HSPC mobilizing agents, and a combination thereof.

10. A method for preparing a bio-nanoparticle for delivering an active agent into a hematopoietic stem & progenitor cell (HSPC), comprising coating a core with a biological membrane at an effective weight ratio for forming a bio-nanoparticle, wherein the core comprises the active agent and a polymer, wherein the biological membrane comprises two layers of phospholipids and one or more surface proteins of a megakaryocyte (Mk), and wherein the active agent remains active after being delivered into the HSPC.

11. The method of claim 10, further comprising preparing the biological membrane from a megakaryocyte (Mk), megakaryocytic microparticle or megakaryocytic extracellular vesicle.

12. The method of claim 11, further comprising preparing the megakaryocyte (Mk), megakaryocytic microparticle or megakaryocytic extracellular vesicle from a hematopoietic stem & progenitor cell (HSPC) or a human megakaryocyte cell line.

13. The method of claim 10, further comprising preparing the biological membrane from a megakaryocyte (Mk) after one or more components of the Mk are removed from the Mk, wherein the one or more components are selected from the group consisting of cytosolic, nuclear and mitochondrial components.

14. The method of claim 10, further comprising adhering the biological membrane to the core by an electrostatic interaction.

15. The method of claim 10, wherein the one or more surface proteins are selected from the group consisting of CD62P, VLA-4 (CD49d), CD41, CD150, CXCR4,thrombopoietin (TPO) receptor, c-kit, CD34, CD105 (endoglin), CD31 (9PECAM-1), JAM-A, Tie-2, KDR (VEGF receptor 2) and a combination thereof.

16. The method of claim 10, wherein the polymer is poly(lactic-co-glycolic acid) (PLGA).

17. The method of claim 10, wherein the active agent is hydrophobic, further comprising preparing the core from a single-emulsion or double emulsion.

18. The method of claim 10, wherein the active agent is selected from the group consisting of an imaging agent, a therapeutic agent, and a combination thereof, wherein the imaging agent is selected from the group consisting of fluorophores, MRI contrast agents, CT contrast agents, ultrasound contrast agents, and a combination thereof, wherein the therapeutic agent is a nucleic acid molecule selected from the group consisting of siRNA, miRNA, DNA, and a combination thereof, wherein the DNA is a single-stranded DNA, wherein the therapeutic agent is selected from the group consisting of chemotherapeutics, HSPC mobilizing agents, and a combination thereof, and wherein the therapeutic agent is a chemotherapeutic.

19. A method for treating a disease or condition in a subject in need thereof, comprising administering to the subject an effective amount of the bio-nanoparticles of claim 1.

20. The method of claim 19, wherein the disease or condition is selected from the group consisting of bone marrow failure disorder, leukemia, lymphoma, multiple myeloma, aplastic anemia, sickle cell disease, thalassemia, autoimmune disorders, HIV, multiple sclerosis, myeloproliferative disorder, myelodysplastic syndrome, and other forms of cancer.
